(19) United States
(12) Patent Application Publication
(10) Pub. No.: US 2024/0114944 A1
(43) Pub. Date: Apr. 11, 2024

(54) METHOD FOR PRODUCING SLOWLY DIGESTIBLE BRANCHED STARCH HYDROLYSATES AND USES THEREOF

(71) Applicant: ROQUETTE FRERES, Lestrem (FR)

(72) Inventors: Bernard PORA, Hazebrouck (FR); Jovin HASJIM, Hauts-de-France (FR); Shiyao YU, Shanghai, Putuo District (CN)

(21) Appl. No.: 18/260,183

(22) PCT Filed: Dec. 28, 2021
(86) PCT No.: PCT/EP2021/025518

§ 371 Date: Jun. 30, 2023

(30) Foreign Application Priority Data
Jan. 7, 2021 (CN) 202110019000.6
Jan. 8, 2021 (EP) 21305019.8

(51) Int. Cl. A23L 29/30; C08B 30/18

(52) U.S. Cl. CPC A23L 29/35; C08B 30/18

(57) ABSTRACT

The present invention relates to a composition comprising slowly digestible branched starch hydrolysates, methods for producing said compositions and uses thereof, in particular in the food industry.

### FIELD OF THE INVENTION

The present invention relates to a composition comprising slowly digestible branched starch hydrolysates, methods for producing said compositions and uses thereof, in particular in the food industry.

### PRIOR ART

Carbohydrates are the main source of energy in human and animal food and are important constituents of a balanced and healthy diet.

Amongst them, starch represents an important component in human nutrition. Starch is a polysaccharide, produced by most green plants as energy storage. It is the most common carbohydrate in human diets and is contained in large amounts in staple foods, such as potato, maize (corn), rice, wheat and cassava.

Starch is a complex carbohydrate, made up of amylose and amylopectin molecules.

Amylose is a polysaccharide made of α-D-glucose units, bound to each other through α-1,4 glycosidic bonds with a few branches. It makes up approximately 20-30% of normal starches, but the ratio differs from 0% to 99% according to the botanical origins of the starches and the mutation on the plant genes.

Amylopectin is highly branched polymer of α-glucose units linked in a linear way with α-1,4 glycosidic bonds. Branching takes place with α-1,6 bonds, which is about 4 to 5% of the total glycosidic bonds.

From a nutritional point of view, starch can be classified into three nutritional fractions: rapidly digestible starch (RDS), slowly digestible starch (SDS), and resistant starch (RS). SDS has drawn recent interest, because foods containing SDS are considered to have a low glycemic index (GI), providing a long-lasting glucose release. This is particularly useful for individuals wishing to control the rate of glucose release into the bloodstream, such as diabetic or prediabetic patients. It can also be used to prevent hyperinsulinemia-induced hypoglycemia and hunger as well as to improve cognitive functions throughout the day.

The amount of SDS is generally determined using an in vitro method developed by Englyst et al., and published in 1992 in the European Journal of Clinical Nutrition, volume 46, pp. S33-S50.

Attempts have been made in the art to increase the amount of SDS in starch-based compositions by increasing the branching density, i.e. by increasing the number of α-1,6 glycosidic bonds. The two main enzymes for starch digestion in the human body is α-amylase and glucoamylase. The α-1,6 glycosidic bonds cannot be hydrolyzed by α-amylase and, in addition, the branching points prevents the binding of the starch substrate to α-amylase. Although glucoamylase can hydrolyze α-1,6 glycosidic bonds, it is an exoenzyme and its hydrolytic rate is slower than α-amylase, which is an endoenzyme. Therefore, having a higher number of α-1,6 glycosidic bonds can reduce the digestion rate of the starch.

Lee et al. (PLOSOne, 2013, 8(4), e59745, “Enzyme-Synthesized Highly Branched Maltodextrins Have Slow Glucose Generation at the Mucosal a-Glucosidase Level and Are Slowly Digestible In Vivo”) describes the sequential action of starch branching enzyme and β-amylase on waxy corn starch. Dialysis was used to remove the small sugars and oligosaccharides.

Shi et al. (Food Chemistry, 2014, 164, pp 317-323 “Pea starch (Pisum sativum L.) with Slow Digestion Property Produced Usingβ-Amylase and Transglucosidase”) describes the treatment of pea starch by β-amylase with or without transglucosidase. Ethanol precipitation was used to remove small sugars and oligosaccharides.

In both papers, slower digestion properties were obtained. However, since the starting material was starch, the processes described in these papers are not cost effective for scaling up to industrial processes because the high viscosity of starch prevents the use of compositions at sufficient solid contents. In addition, the high water content will lead to the high hydrolytic reaction of starch by the enzymes, resulting in low product yield.

Patent document US2011/0020496 A1 describes a method for producing a branched dextrin by allowing maltose-generating amylase, such as β-amylase, and transglucosidase in an enzyme unit ratio of 2:1 to 44:1, respectively, to act on an aqueous dextrin solution. The high amount/ratio of β-amylase (up to 11.9 U/g substrate with the optimum concentration at 6.3 U/g) was intended to hydrolyze the outer α-1,4 linkages of the dextrin, reducing the number of α-1,4 linkages and thus increasing the degree of branching. However, based on the in vitro digestion results, there was only less than 15% reduction in the released glucose between the branched dextrin and the control dextrin (substrate) after 2-hour digestion. In addition, their amount of transglucosidase might be too low for an effective branching reaction, and their high amount/ratio of β-amylase could generate a high amount of free maltose.

Therefore, none of the prior art documents provide a large-scale method for preparing a composition of branched, slowly digestible starch hydrolysates that contain minimal amounts of sugars with the degree of polymerization (DP) of 1 to 3 (or high yield of branched starch hydrolysates). There is still a need in the art for slow release of glucose to prolong satiety, endurance workout and cognitive functions.

The Applicant has thus, to its credit, developed such a composition and its manufacturing process, which will be disclosed in more details below.

### SUMMARY OF THE INVENTION

The present invention relates to a method for preparing a composition comprising branched starch hydrolysate, comprising the step of incubating a composition comprising starch hydrolysate with a mixture of maltose-generating amylase and transglucosidase enzymes in unit ratio of 9:5 to 1:20, advantageously 9:5 to 1:10, preferably 10:6 to 1:2, even more preferably around 3:2 to 1:1.

The invention also relates to a composition comprising branched starch hydrolysate obtainable by the method defined above.

The invention further relates to a composition comprising branched starch hydrolysate wherein said composition:
a) has a DE value between 10 and 50, preferably between 12 and 30, even preferably between 15 and 25,
b) has a percentage of α-1,6 linkages of at least 7, preferably of at least 8%, at least 9%, at least 10%, at least 11%, at least 12%, at least 13%, at least 14%, at least 15%, at least 16%, at least 17%, at least 18%, at least 19%, preferably of at least 20%, at least 21%, at least 22%, at least 23%, at least 24%, at least 25%, and even more preferably of at least 30%,
c) comprises at least 30% by weight, preferably at least 35%, preferably at least 40% by weight, even more preferably at least 43% by weight of branches of the branched starch hydrolysate with a DP equal to or greater than 10.

Another object of the present invention is the use of a composition comprising branched starch hydrolysate as defined above in food formulations.

The invention also provides food formulations containing a composition comprising branched starch hydrolysate as defined above.

The invention also relates to a kit for preparing a composition of branched starch hydrolysates comprising a mixture of maltose-generating amylase and transglucosidase enzymes in unit ratio of 9:5 to 1:20, advantageously 9:5 to 1:10, preferably 10:6 to 1:2, even more preferably around 3:2 to 1:1.

### DETAILED DESCRIPTION

A first object of the present invention relates to a method for preparing a composition comprising branched starch hydrolysate, comprising the step of incubating a composition comprising starch hydrolysate with a mixture of maltose-generating amylase and transglucosidase enzymes in enzymatic activity unit ratio of 9:5 to 1:20, advantageously 9:5 to 1:10, preferably 10:6 to 1:2, even more preferably around 3:2 to 1:1.

The inventors have surprisingly shown that it is possible to carry out a large-scale production process for branched starch hydrolysates, which possess slow or low digestibility, high solubility, low viscosity, good cold stability, and/or low to no dietary fibers.

The inventors have shown that specific ratios between β-amylase and transglucosidase can be used to treat a composition comprising starch hydrolysate in order to increase the degree of branching in said starch hydrolysate, thereby lowering and/or slowing down the digestibility of said composition.

As used herein, the term “branched” refers to the degree of branching, indicated by the percentage of α-1,6 glycosidic bonds to the total glycosidic bonds, higher than that in the native starch molecules (about 4 to 5%).

Without wishing to be bound by theory, it is believed that the specific ratios between the enzymes favor the branching reaction between maltose and maltodextrin. In particular, a small amount of β-amylase results in small amounts of free maltose being generated throughout the enzymatic branching process, thereby reducing the transglucosidase reaction between two maltose molecules and decreasing the amount of isomaltose, panose, and other isomaltooligosaccharides (IMOS) by-products

The high solid content also favors the branching reaction of transglucosidase over other enzymatic reactions (such as hydrolysis) and results in a more efficient production of branched starch hydrolysates with less undesirable products.

Advantageously, the method of the invention results in the production of small amounts of sugars of DP 1 to 3. No additional molecular separation step, such as ethanol precipitation, membrane filtration, dialysis, and chromatographic separation, is required in order to remove these undesirable by-products and the composition can be used as such. However, these small amounts of sugars can be removed by the methods mentioned above.

For the purposes of the present invention, “food product” is intended to mean a formulation or composition that can be ingested by an animal or a human being. Examples of food products include foodstuffs for human consumption, animal feeds, and beverages.

In the present invention, the term “starch hydrolysate” refers to any product obtained by enzymatic or acid, preferably enzymatic hydrolysis of starch from legumes, cereals or tubers. Various hydrolysis processes are known and have been generally described on pages 511 and 512 of the book Encyclopedia of Chemical Technology by Kirk-Othmer, 3rd Edition, Vol. 22, 1978. These hydrolysis products are also defined as purified and concentrated mixtures of molecules made up of D-glucose polymers essentially bound by α-1,4 glycosidic linkages with about 4 to 5% of branching points formed by α-1,6 glycosidic bonds, having a wide range of molecular weights, and they are completely soluble in water. Starch hydrolysates are very well known and perfectly described in Encyclopedia of Chemical Technology by Kirk-Othmer, 3rd Edition, Vol. 22, 1978, pp. 499 to 521.

In a preferred embodiment, the starch hydrolysate of the invention is not obtained by acid treatment of starch.

In one embodiment, the starch hydrolysate useful for the invention is obtained by enzymatic treatment of starch with α-amylase.

Thus, in the present invention, the starch hydrolysis product is selected from starch, gelatinized starch, maltodextrins, glucose syrups, maltose and any mixtures thereof.

The distinction between starch hydrolysis products is mainly based on the measurement of their reducing power, conventionally expressed by the concept of “dextrose equivalent” or DE. The DE corresponds to the quantity of reducing sugars, expressed in dextrose equivalent per 100 g of dry matter of the product. DE therefore measures the intensity of starch hydrolysis, since the product is hydrolyzed to a greater extent; the more small molecules it contains (such as dextrose and maltose for example) and the higher its DE value is. On the contrary, the more large molecules the product contains (polysaccharides), the lower its DE value is.

From a regulatory point of view, and also within the meaning of the present invention, maltodextrins have a DE of from 1 to 20, and the glucose syrups have a DE of more than 20. Such products are for example maltodextrins and dehydrated glucose syrups sold by the Applicant under the names of GLUCIDEX® (DE available=1, 2, 6, 9, 12, 17, 19 for maltodextrins and DE=21, 29, 33, 38, 39, 40, 47 for glucose syrups). Mention may also be made of the glucose syrups sold by the Applicant under the name “Roquette glucose syrups”.

In one embodiment, the composition comprising starch hydrolysate is obtained from a starch selected from tapioca, waxy tapioca, maize, waxy maize, rice, waxy rice, wheat, waxy wheat, barley, waxy barley, sorghum, waxy sorghum, potato, waxy potato, sweet potato, waxy sweet potato, sago, millet, mung bean, pea, faba bean, chickpea, arrowroot, buckwheat, quinoa, lotus root, preferably tapioca, waxy maize, or pea.

In a preferred embodiment, said starch is tapioca starch.

In a preferred embodiment, the starch hydrolysate is obtained by incubating said starch source with one or multiple amylases, such as α-amylase, preferably thermostable α-amylase.

The DE of the starch hydrolysate can be between 1 and 30, preferably between 5 and 20, even more preferably between 8 and 15. Generally, starch hydrolysates having a DE of less than 20 are considered as maltodextrins.

Any suitable method described in the art can be used to determine the DE value of a product. Typically, the DE value can be determined according to the method of Bertrand (Bulletin de la Société Chimique de France, 1906, 35 pp. 1285-1299).

In a preferred embodiment, the starch hydrolysate comprises less than 10%, preferably less than 9%, less than 8%, less than 7%, less than 6%, less than 5%, less than 4%, even more preferably less than 3%, less than 2% by weight of total dietary fiber or does not comprise dietary fiber.

The free maltose content of the starch hydrolysate can be less than 15%, preferably less than 10%, even more preferably less than 5% by weight. Typically, the maltose content can be measured using chromatography, such as high-performance liquid chromatography (HPLC) and high-performance anion-exchange chromatography (HPAEC). A preferred method for determining the amount of free maltose is HPAEC, as detailed in Examples 4 to 6 below.

In one embodiment, the viscosity of the composition comprising starch hydrolysate prior to branching reaction is below 5000 cP, more preferably below 2000 cP, at 50% solid content at 25° C. Methods for measuring the viscosity of starch hydrolysate compositions are known in the art. Typically, the viscosity can be measured using laboratory viscometer (AMETEK Brookfield) or Rapid Visco Analyser (RVA, Perten Instruments).

Advantageously, in one embodiment of the invention, the composition comprising starch hydrolysate has a solid content comprised between 30% and 70% by weight of the total composition, preferably between 40% and 60% by weight of the total composition, even more preferably between 45% and 55% by weight of the total composition.

The high solid content favors the branching reaction of transglucosidase over other enzymatic reactions (such as hydrolysis) and results in a more efficient production of branched starch hydrolysates with less undesirable by-products.

This also allows the method of the invention to be carried out at industrial scales, in order to obtain high production efficiency. In contrast, many of the methods described in the art, especially those that use starch as a substrate rather than starch hydrolysates, are only suited for laboratory-scale experiments.

According to the method of the invention, the step of incubating the composition comprising starch hydrolysate with a mixture of maltose-generating amylase and transglucosidase enzymes is carried out at a temperature comprised between 40° C. and 70° C., preferably between 50° C. and 60° C., even more preferably around 55° C.

This enzymatic branching reaction step is carried out for a time period sufficient to allow the generation of branched starch hydrolysate in a suitable amount. Typically, the step of incubating the composition comprising starch hydrolysate with a mixture of maltose-generating amylase and transglucosidase enzymes is carried out for a duration comprised between 1 and 72 hours, preferably between 2 and 24 hours, even more preferably 4 to 8 hours.

Typically, the step of incubating the composition comprising starch hydrolysate with mixture of maltose-generating amylase and transglucosidase enzymes can be carried out at pH value comprised between 4 and 8, preferably between 4.5 and 7, even more preferably between 5 and 6.5.

After the enzymatic treatment step, the reactions are stopped by enzymatic deactivation, such as incubation at 95° C. for 30 minutes.

The method of the invention requires simultaneous treatment by maltose-generating amylase and transglucosidase enzymes in a given unit ratio.

As used herein, one unit of maltose-generating amylase is defined as the amount of enzyme to generate 1 μmol of maltose in one minute using 5% by weight waxy maize starch maltodextrin aqueous solution having a DE of around 12 (such as GLUCIDEX 12C, Roquette) as a substrate under reaction conditions with pH 5.5 and a reaction temperature of 55° C.

In one embodiment of the invention, the maltose-generating amylase is selected from the group consisting of maltogenic α-amylases and maltogenic β-amylases, preferably β-amylase, even more preferably wheat β-amylase.

Typically, said wheat β-amylase can be used at a concentration of 1.8 units per g of solid content in the composition comprising starch hydrolysate.

As used herein, one unit of transglucosidase is defined as an enzyme ability to generate 1 mol of glucose in one minute using 1% by weight methyl-α-D-glucopyranoside aqueous solution as a substrate under reaction conditions with pH 5.5 and a reaction temperature of 55° C.

According to a preferred embodiment of the invention, the transglucosidase is a D-glucosyltransferase (E.C.2.3.1.24). In one embodiment, the transglucosidase is selected from the group consisting of transglucosidase L “Amano” ® (commercialized by Amano Enzyme), transglucosidase L-2000@ (commercialized by DuPont) and Branchzyme® (commercialized by Novozymes).

The simultaneous use of maltose-generating amylase and transglucosidase in defined unit ratios allows a precise control of the amount of free maltose generated throughout the reaction. The concentration of free maltose can be monitored during and after the incubation. Typically, the concentration of free maltose can be less than 40%, preferably less than 30% even more preferably less than 20% by weight of the sugar composition. In a preferred embodiment, the concentration of free maltose is less than 12%, preferably less than 11%, less than 10%, less than 9%, less than 8%, even more preferably less than 7%, less than 6% or less than 5% by weight of the composition.

The present invention also relates to a composition comprising branched starch hydrolysate obtainable by the method as defined above.

In one aspect, the invention relates to a composition comprising branched starch hydrolysate wherein said composition:
a) has a DE value between 10 and 50, preferably between 12 and 30, even preferably between 15 and 25

and/or
b) has a percentage of α-1,6 linkages of at least 7, preferably of at least 8%, at least 9%, at least 10%, at least 11%, at least 12%, at least 13%, at least 14%, at least 15%, at least 16%, at least 17%, at least 18%, at least 19%, preferably of at least 20%, at least 21%, at least 22%, at least 23%, at least 24%, at least 25%, and even more preferably of at least 30%,

and/or
c) comprises at least 30% by weight, preferably at least 35%, preferably at least 40% by weight, even more preferably at least 43% by weight of branches of the branched starch hydrolysate with a DP equal to or greater than 10.

Typically, the DE value or “dextrose equivalent” value is determined according to the method of Bertrand (Bulletin de la Société Chimique de France, 1906, 35 pp. 1285-1299).

As used herein, the expression “percentage of α-1,6 linkages” refers the degree of branching of the branched starch hydrolysate. It is calculated as the amount of α-1,6 glycosidic linkages divided by the sum of α-1,4 and α-1,6 glycosidic linkages.

Any suitable method can be used to determine the percentage of α-1,6 linkages. Typically, the amounts of α-1,4 and α-1,6 glycosidic linkages can be analyzed using proton nuclear magnetic resonance (H NMR) as described in Example 4 of the “Examples” Section below. The composition according to the present invention is also characterized by the percentage (by weight) of branches that have a degree of polymerization (DP) of at least 10. This DP is calculated based on the total weight of linear starch hydrolysate, i.e. after the starch hydrolysate has been debranched by enzymatic treatment with an isoamylase (which hydrolyzes the α-1,6 glycosidic linkages).

The DP of the branches (or linear molecules) can be calculated by any suitable method in the art. Typically, the DP of the linear starch hydrolysate can be estimated from the hydrodynamic radius using the Mark-Houwink equation following the method of Liu et al. (Macromolecules, 2010, 43, pp. 2855-2864).

Advantageously, the inventors have demonstrated that the composition comprising branched starch hydrolysate according to invention is slowly digestible. The term “slowly digestible” as used herein refers to a branched starch hydrolysate composition that contains a higher fraction of SDS and RS as measured by the Englyst method than the starch hydrolysate compositions that have not been subjected to the enzymatic treatment step of the invention. Typically, a composition is deemed “slowly digestible” if the sum of SDS and RS fractions measured according to the method of Englyst et al. (European Journal of Clinical Nutrition, 1992, 46, pp. S33-550) shows a 50% increase, preferably a 100% increase, even more preferably a 150% increase to the original sum of SDS and RS of starch hydrolysates before incubation with transglucosidase and maltose-generating amylase.

Alternatively, a composition is deemed “slowly digestible” if the digestion rate as measured according to the method of Yu et al. (Food Chemistry, 2018, 264, pp. 284-292) is less than 80%, preferably less than 60%, even more preferably less than 50% of the digestion rate of the starch hydrolysates before incubation with transglucosidase and maltose-generating amylase.

The methods of Englyst et al. and Yu et al. are carried out as described in Example 1 of the “Example” Section below.

In a preferred embodiment, the composition comprising branched starch hydrolysate according to invention is stable against retrogradation.

The term “stable against retrogradation” refers to a composition that does not (or to a lesser extent) undergo the reorganization process known as retrogradation wherein the molecules within a gelatinized starch paste re-associate to form more ordered structure. This stability against retrogradation is reflected by a smaller increase in the viscosity after cold storage and a smaller change in the gel transparency after cold storage as compared to starch hydrolysate compositions that have not been subjected to the enzymatic branching treatment step of the invention.

In a preferred embodiment, the composition comprising branched starch hydrolysate according to invention comprises no or low dietary fiber.

A composition is deemed to comprise “low dietary fiber” if it comprises less than 10%, preferably less than 9%, less than 8%, less than 7%, less than 6%, less than 5%, less than 4%, even more preferably less than 3%, less than 2% by weight of dietary fiber.

The amount of dietary fiber can be determined by any suitable method known in the art. Typically, the skilled person can carry out the AOAC Method 2009.01 to determine the amount of total dietary fibers.

The invention also relates to a kit for preparing a composition of branched starch hydrolysates comprising a mixture of maltose-generating amylase and transglucosidase The invention will be understood more clearly on reading the examples which follow, which are intended to be purely illustrative and do not in any way limit the scope of the protection. The term of “maltodextrin” is used loosely in the examples to represent starch hydrolysate regardless of their DE.

### EXAMPLES

Transglucosidase L-2000@ (from DuPont): Activity was quantified using the method described above and is equal to 1598 U/mL (around 1.6 U/μL).

Transglucosidase L “Amano” ® (from Amano Enzyme): Activity was quantified using the method described above and is equal to 1077 U/mL (around 1.1 U/μL).

wheat β-amylase (Roquette): Activity was quantified using the method described above and is equal to 17906 U/mL (around 1.8 U/0.1 μL).

### Example 1: Enzymatic Branching Modification of Tapioca Maltodextrins Using Transglucosidase L-2000® (from DuPont)

### Preparations

Tapioca Maltodextrins

Spezyme® Cassava (a thermostable α-amylase from DuPont) was added to 40% tapioca starch slurry (using deionized water) at a concentration of 0.21 or 0.42 μL/g dry starch. The mixture was autoclaved for 5 min and allowed to cool in the autoclave for additional 30 min. The enzyme was finally deactivated by adjusting the pH of the slurry to 3.0-3.5 using 5% HCl solution. After 30 min, the pH was neutralized back to 6.0 using 1M and 0.1M NaOH solution. The solution was then spray dried to produce maltodextrin powder. The resulting maltodextrin samples were labelled as TM21N and TM42N, respectively, were used for further enzymatic branching reaction according to the present invention.

Branched Tapioca Maltodextrins

A 45% w/v tapioca maltodextrin solution containing 5% w/v glycerol was prepared. The glycerol served as an internal standard for sugar composition analysis using HPLC. The maltodextrin was solubilized by constant stirring in a water bath at 55° C. for 60 min. Transglucosidase L-2000® (from DuPont) and wheat β-amylase (Roquette) were added at the concentrations of 1 μL (or 1.6 U) and 0.1 μL (or 1.8 U) per gram dry maltodextrin, respectively. The mixture was incubated at 55° C. Samples were collected after 4-hour reaction (labelled as TM21H4 and TM42H4 derived from TM21N and TM42N, respectively) and after 24-hour reaction (labelled as TM21H24 and TM42H24 derived from TM21N and TM42N, respectively). The enzymes were deactivated by heating the samples in boiling water for 20 min.

### Analysis

Dextrose Equivalent

The DE values of the unmodified and the branched tapioca maltodextrins were analyzed and presented in Table 1.

TABLE 1
DE values and maltose contents of unmodified
and branched tapioca maltodextrins.

| | | Maltose content |
|---|---|---|
| Sample | DE value | (% by weight) |
| TM21N | 12 | 2.69 |
| TM21H4 | 13 | 9.14 |
| TM21H24 | 16 | 4.90 |
| TM42N | 17 | 4.73 |
| TM42H4 | 17 | 11.27 |
| TM42H24 | 21 | 5.78 |

The tapioca maltodextrin samples produced using Spezyme Cassava at the concentrations of 0.21 and 0.42 μL/g dry starch had DE values of 12 and 17, respectively. The enzymatic branching reaction according to the present invention increased the DE values up to 16 and 21, respectively.

Maltose Content

Samples were diluted to ˜1% solid content using ultrapure water and then filtered through 0.45-μm syringe filter before injecting into a HPLC system (Alliance e2695 Separations Module, Waters) equipped with a refractive index (RI) detector (2414, Waters). The injection volume was 100 μL. The columns consisted of precolumn (TSK guard column PWXL, 60 mm i.d. 4 cm) and two LC columns (TSK-GEL G2500PWXL, 7.8 mm i.d. 30 cm) connected in series. The columns were placed inside a column oven at 80° C. Ultrapure water was used as the mobile phase at a flow rate of 0.5 mL/min.

Table 1 shows the amount of maltose in the tapioca maltodextrin samples increased after 4-hour enzymatic branching reaction using transglucosidase and β-amylase, and then decreased after 24-hour branching reaction. The increase was due to the hydrolysis reaction of β-amylase, increasing the DE values. The decrease in maltose content after 24-hour branching reaction was due to the reaction of tranglucosidase that used maltose as the substrate.

Viscosity

Maltodextrin solutions were evaporated to a solid content of about 50%. The solutions were heated in boiling water bath and stored in a refrigerator. On the second day, the viscosity of each solution (25 g) was analyzed using an RVA (RVA 4500, Perten Instruments) with the heating profile summarized in Table 2. The solutions were stored in the refrigerator at 4° C. for additional one month and then reanalyzed using the RVA following the same heating profile.

TABLE 2
RVA heating profile for viscosity measurement
at different temperatures.

| Time | Temperature (° C.) | Speed (rpm) |
|---|---|---|
| 00:00:00 | 20 | 960 |
| 00:00:10 | 20 | 160 |
| 00:01:00 | 20 | 160 |
| 00:36:00 | 90 | 160 |
| 00:40:00 | 90 | 160 |

TABLE 3
Viscosity increases between after 1-day and after 1-month cold
storage of unmodified and branched tapioca maltodextrins.

| | | | Viscosity | | |
|---|---|---|---|---|---|
| Sample | Solid content (%) | Condition | 20° C. | 30° C. | 40° C. |
| TM21N | 49.3 | After 1-day storage | 1367 cP | 1059 cP | 677 cP |
| | | After 1-month storage | 1988 cP | 1466 cP | 904 cP |
| | | Increase | 45% | 38% | 34% |
| TM21H4 | 52.7 | After 1-day storage | 613 cP | 462 cP | 290 cP |
| | | After 1-month storage | 793 cP | 579 cP | 359 cP |
| | | Increase | 29% | 25% | 24% |
| TM21H24 | 53.1 | After 1-day storage | 344 cP | 258 cP | 171 cP |
| | | After 1-month storage | 459 cP | 336 cP | 218 cP |
| | | Increase | 33% | 30% | 27% |
| TM42N | 50.7 | After 1-day storage | 834 cP | cP cP | 343 cP |
| | | After 1-month storage | 1706 cP | 1168 cP | 741 cP |
| | | Increase | 105% | 116% | 116% |
| TM42H4 | 53.1 | After 1-day storage | 370 cP | 283 cP | 190 cP |
| | | After 1-month storage | 610 cP | 458 cP | 296 cP |
| | | Increase | 65% | 62% | 56% |
| TM42H24 | 51.2 | After 1-day storage | 205 cP | 143 cP | 107 cP |
| | | After 1-month storage | 255 cP | 195 cP | 137 cP |
| | | Increase | 24% | 36% | 28% |

The increases in the viscosities between after 1-day and after 1-month cold storage are more obvious at low temperature. Table 3 shows the viscosity increases between the two periods of cold storage (1 day and 1 month) measured at 20, 30, and 40° C. The differences became smaller after the enzymatic branching reaction according to the present invention. In addition, TM211H24 had similar DE value to TM42N and TM42H4, which were about 16-17, but the viscosity increase of TM211H24 was smaller. The smaller viscosity increase indicated that the enzymatic branching reaction according to the present invention stabilized the tapioca maltodextrin solutions against retrogradation during cold storage. The branched tapioca maltodextrin solutions were also more transparent after cold storage than the unmodified tapioca maltodextrin solutions (data not shown).

In Vitro Starch Digestibility

Two in vitro digestion methods were used to access the digestibility of the branched tapioca maltodextrins.

The first method was based on the method of Englyst et al. (European Journal of Clinical Nutrition, 1992, 46, pp. S33-S50), which was as follows.

Acetate buffer (0.1 M) was prepared by dissolving 13.6 g sodium acetate trihydrate in 250 mL saturated benzoic acid solution, diluting it to 1 L with deionized water, adjusting the pH to 5.2 using 0.1 M acetic acid, and adding 4 mL 1 M CaCl₂ per liter of buffer.

Enzyme solution was prepared fresh before the experiments. Four 50-mL centrifuge tubes were prepared where each containing 3.0 g porcine pancreatin (P1750, Sigma) mixed with 20 mL water. The mixture was stirred for 10 min and centrifuged for 10 min at 1500×g. The supernatants (13.5 mL from each tube) were combined and mixed with 2.8 mL amyloglucosidase (A7095, Sigma) and 7.95 mL deionized water.

Each sample (1.00 g, dry basis) was mixed with 20 mL 0.1 M acetate buffer and 50 mg guar gum in a 50-mL tube. A blank was prepared using 20 mL 0.1 M acetate buffer and 50 mg guar gum, without sample. The samples and the blank were equilibrated at 37° C. in a water bath with shaking. Taking one tube per minute, 5 mL enzyme solution was added to the samples and the blank. Immediately after mixing, the tubes were returned to the water bath at 37° C. for 120 min with shaking. An aliquot (0.25 mL at 0 min and 0.20 mL at 20 min and 120 min) was transferred from each tube to a 15-mL tube containing 10 mL 66% v/v ethanol solution and mixed well. The ethanol solutions were centrifuged at 1,500×g to obtain the clear supernatant, and the glucose content in each supernatant (0.1 mL) was analyzed using D-glucose assay kit GOPOD format (Megazyme). The weight percentage of glucose released after 0, 20, and 120 min in vitro digestion were labelled as free glucose (FG), G₂₀, and G₁₂₀, respectively. The RDS, SDS, and RS were calculated as follows:

RDS=(G₂₀−FG)×0.9

SDS=(G₁₂₀−G₂₀)×0.9

RS=100%−(FG+RDS+SDS)=100%−(G₁₂₀×0.9)

TABLE 4
In vitro digestibilities of unmodified and branched tapioca
maltodextrins following the method of Englyst et al.

| | Free | Rapidly | Slowly | |
|---|---|---|---|---|
| | glucose | digestible | digestible | Resistant |
| Samples | (%) | starch (%) | starch (%) | starch (%) |
| TM21N | 0.3 | 91.8 | 2.1 | 5.9 |
| TM21H4 | 5.8 | 66.0 | 5.5 | 22.7 |
| TM21H24 | 13.6 | 50.0 | 3.0 | 33.4 |
| TM42N | 0.3 | 93.3 | 0.1 | 6.4 |
| TM42H4 | 4.8 | 69.1 | 3.1 | 23.0 |
| TM42H24 | 13.6 | 51.4 | 6.2 | 28.7 |

The in vitro digestibilities of the branched tapioca maltodextrins measured by the method of Englyst et al. showed that the enzymatic branching treatment with transglucosidase and β-amylase according to the present invention resulted in a greater proportion of the sum of SDS and RS, while reducing the percentage of RDS (Table 4).

The second in vitro digestion method was based on the method of Yu et al. (Food Chemistry, 2018, 264, pp. 284-292) with a non-linear least-square (NLLS) fitting. In brief, each sample accurately weighed to 50 mg in a 15-mL centrifuge tube and mixed with 2 mL deionized water and 8 mL enzyme solution. The enzyme solution was prepared fresh on the day by mixing 4 mg pancreatin (P1750, Sigma) and 0.2 mL amyloglucosidase (E-AMGDF, Megazyme) in 96 mL 0.2 M sodium acetate buffer (pH 6.0 containing 200 mM calcium chloride, 0.49 mM magnesium chloride, and 0.02% sodium azide). The samples were incubated at in a water bath at 37° C. and a stirring speed of 300 rpm. Aliquots (0.1 mL) were collected at different time points between 0 to 300 min and each was transferred to a 1.5-mL micro-centrifuge tube containing 0.9 mL absolute ethanol. The ethanol solutions were centrifuged at 1,500×g to obtain the clear supernatant, and the glucose content in each supernatant (0.1 mL) was analyzed using D-glucose assay kit GOPOD format (Megazyme). The weight percentage of glucose released during in vitro digestion was converted to the percentage of starch hydrolysis by multiplying with a factor of 0.9. The digestion profile was fitted using NLLS to obtain the digestion rate and total digestibility. The former was the slope of the digestion curve and the latter was obtained by extrapolating the digestion time to infinity.

TABLE 5
In vitro digestion rates and total digestibilities of unmodified and
branched tapioca maltodextrins following the method of Yu et al.

| Samples | Digestion rate (1/min) | Total digestibility (%) |
|---|---|---|
| TM21N | 0.0509 | 98.9 |
| TM21H4 | 0.0163 | 78.4 |
| TM21H24 | 0.0236 | 67.4 |
| TM42N | 0.0462 | 96.1 |
| TM42H4 | 0.0145 | 72.3 |
| TM42H24 | 0.0165 | 68.8 |

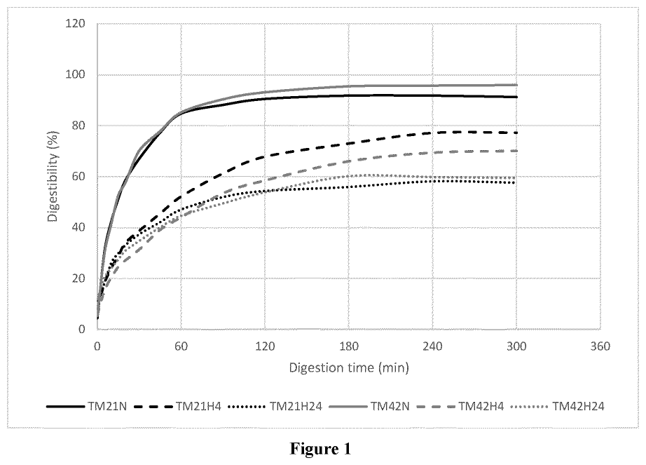

FIG. 1 shows the in vitro digestion curves of the unmodified and the branched tapioca maltodextrins following the method of Yu et al., and Table 5 summarizes the digestion rates and total digestibilities. The branched tapioca maltodextrins prepared using transglucosidase and β-amylase according to the present invention had lower digestion rates and lower total digestibilities than their unmodified tapioca maltodextrin counterparts. The digestion rates of the branched tapioca maltodextrins were less than 50% of those of their unmodified tapioca maltodextrin counterparts. In addition, the total digestibilities of branched tapioca maltodextrins were less than 80% of those of their unmodified tapioca maltodextrin counterparts.

Dietary Fiber Contents

The dietary fiber contents of the unmodified and the branched tapioca maltodextrins were analyzed following the AOAC Method 2009.01 including the HPLC analysis and compared with a resistant dextrin (NUTRIOSE FB06, Roquette).

The results showed that NUTRIOSE FB06, TM42H4, and TM21H24 had 86.65%, 1.65%, and 1.96% dietary fibers, respectively, whereas, other branched tapioca maltodextrins contained negligible amounts of dietary fibers. Essentially branched tapioca maltodextrins contained low or no dietary fibers; the RS fraction observed using the method of Englyst et al. in Table 4 and the indigestible starch observed using the method of Yu et al. in Table 5 could be a fraction of starch that is very slowly digestible.

Molecular Structure

The molecular structures (whole molecular size distribution and chain length distribution) of the branched tapioca maltodextrins according to the present invention were compared with those of the unmodified tapioca maltodextrin and of IMOS (IMO50 and IMO90 from Baolingbao, having DE values of 43 and 42, respectively). IMOS are normally produced from starch using transglucosidase. The whole molecular size and chain length distributions analyses were performed following the method of Gu et al. (Food Chemistry, 2019, 295, pp. 484-492). For the whole molecular size distribution analysis, 2 mg sample was directly dissolved in 1 mL DMSO solution containing 5% LiBr. For chain length distribution, 4 mg sample was first debranched using isoamylase (E-ISAMY, Megazyme) before dissolving into 1 mL DMSO solution containing 5% LiBr. The molecules were separated using a series of SEC columns in series (GRAM pre-column, GRAM 100 and GRAM 1000, PSS) in a LC-20AD system (Shimadzu) coupled with RID-10 A refractive index detector (Shimadzu) with DMSO solution containing 5% LiBr as eluent. The SEC columns were maintained at 80° C. inside a column oven. The weight percentages of molecules were estimated based on the areas under the curve.

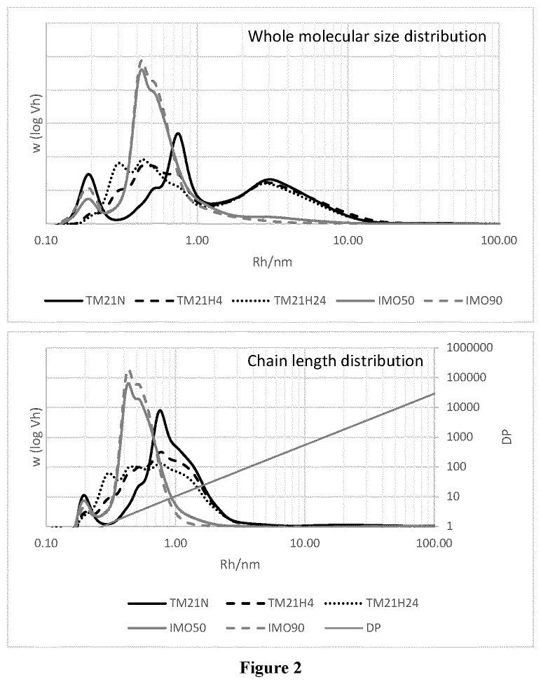

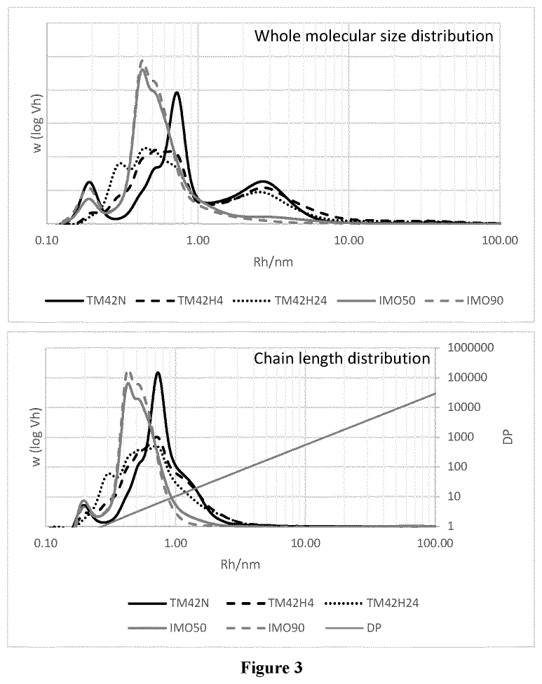

FIGS. 2 and 3 show the results of whole molecular size and chain length distributions (top and bottom, respectively) from the unmodified and the branched tapioca maltodextrins. Table 6 summarizes the weight percentages of whole molecules with hydrodynamic radius (Rₕ) larger and smaller than 1 nm, and branches with DP larger and smaller than 10. The DP of the branches (or linear molecules) was estimated from the hydrodynamic radius using the Mark-Houwink equation following the method of Liu et al. (Macromolecules, 2010, 43, pp. 2855-2864).

TABLE 6
Estimated weight percentages of whole molecules with
hydrodynamic radius (Rₕ) larger and smaller than 1
nm, and branches with DP larger and smaller than 10.

| | Whole molecular | | Chain length distribution | |
|---|---|---|---|---|
| | size distribution | | Rₕ < 1 nm, | Rₕ > 1 nm, |
| Samples | Rₕ < 1 nm, | Rₕ > 1 nm, | DP < 10 | DP > 10 |
| TM21N | 11% | 89% | 39% | 61% |
| TM21H4 | 10% | 90% | 41% | 59% |
| TM21H24 | 12% | 88% | 44% | 56% |
| TM42N | 20% | 80% | 56% | 44% |
| TM42H4 | 11% | 89% | 49% | 51% |
| TM42H24 | 15% | 85% | 57% | 43% |
| IMO50 | 61% | 43% | 87% | 13% |
| IMO90 | 76% | 24% | 96% | 4% |

Both the unmodified and the branched tapioca maltodextrins had more than 80% of their whole molecules with Rₕ larger than 1 nm, whereas less than 50% of the molecules in IMO50 and IMO90 had Rₕ larger than 1 nm. These results agree with their DE values.

The branches of the unmodified and the branched tapioca maltodextrins were also longer than those of the IMOS. More than 40% of the branches in the unmodified maltodextrins and the branched maltodextrins were larger than DP 10, whereas only less than 15% of the branches in IMO50 and IMO90 were larger than DP 10. In addition, IMO50 and IMO90 showed only a small difference in the molecular size distributions before and after debranching, suggesting that IMOS samples were mostly linear and/or not susceptible to isoamylase, whereas the branched tapioca maltodextrins still contained some branches, which were susceptible to isoamylase hydrolysis, as the molecules became smaller after debranching.

Although there is no direct relationship between the hydrodynamic size of a branched molecule to its molecular weight, it can be assumed that most of the small molecules (DP<10) in the whole molecular size distribution were linear and thus the Rh at 1 nm was close to DP 10. Therefore, the enzymatic branching reaction according to the present invention did not reduce the overall molecular size of tapioca maltodextrin to Rh<1 nm or DP<10, and the products were not the same as IMOS.

Conclusions

The enzymatic branching reaction using transglucosidase and β-amylase according to the present invention increased the DE values of tapioca maltodextrins by about 3 after 24 hour reaction time, which could be attributed to the increase in the amounts of small sugars. The branched tapioca maltodextrins were more stable against the retrogradation during cold storage as indicated by the smaller increase in the solution viscosity and opacity after prolonged cold storage. In addition, the branched tapioca maltodextrins showed higher amounts of SDS and RS as analyzed using the method of Englyst et al., and lower digestion rates and total digestibilities as analyzed using the method of Yu et al. However, the branched tapioca maltodextrins essentially contained low or no dietary fibers (less than 2%). The branched tapioca maltodextrins still maintained some branches that were susceptible to isoamylase hydrolysis and most of their molecules were larger than DP 10. Therefore, the branched tapioca maltodextrins are different from IMOS, which are also produced using transglucosidase.

### Example 2: Enzymatic Branching Modification of Waxy Maize Maltodextrin Using Transglucosidase L-2000® (from DuPont)

### Preparations

Branched Waxy Maize Maltodextrin

A 45% w/v waxy maize maltodextrin solution (prepared with deionized water) containing 5% w/v glycerol was prepared using Glucidex 8C (Roquette). The glycerol served as an internal standard for sugar composition analysis using HPLC. The maltodextrin was solubilized by constant stirring in a water bath at 55° C. for 60 min. Transglucosidase L-2000® (from DuPont) and wheat β-amylase (Roquette) were added at the concentrations of 1 μL (or 1.6 U) and 0.1 μL (or 1.8 U) per gram dry maltodextrin, respectively. The mixture was incubated at 55° C. Samples were collected after 1-, 2-, 4-, and 24-hour reaction (labelled as G8CH1, G8CH2, G8CH4 and G8CH24, respectively, and the unmodified Glucidex 8C was labelled as G8CN). The enzymes were deactivated by heating the samples in boiling water for 20 min.

Analysis

Dextrose Equivalent

The DE values of the unmodified and the branched waxy maize maltodextrins were analyzed and presented in Table 7.

TABLE 7
DE values and maltose contents of unmodified
and branched waxy maize maltodextrins.

| | | Maltose content |
|---|---|---|
| Sample | DE value | (% by weight) |
| G8CN | 8 | 1.46 |
| G8CH1 | 6 | 2.94 |
| G8CH2 | 7 | 2.90 |
| G8CH4 | 7 | 2.88 |
| G8CH24 | 8 | 3.03 |

The DE values did not show obvious changes after the enzymatic branching reaction using transglucosidase and β-amylase according to the present invention.

Maltose Content

The maltose contents of the unmodified and the branched waxy maize maltodextrins were analyzed using the same HPLC method as in Example 1.

Table 7 shows the amount of maltose in the waxy maize maltodextrin sample increased after the enzymatic branching reaction using transglucosidase and β-amylase. This was due to the hydrolysis reaction of β-amylase; however, the increase in the maltose content did not show an obvious increase in the DE values of waxy maize maltodextrin.

Viscosity

The viscosity after cold storage of the unmodified and the branched waxy maize maltodextrins were analyzed using the same RVA method as in Example 1.

TABLE 8
Viscosity Increases between after 1-day and after 1-month cold
storage of unmodified and branched waxy maize maltodextrins.

| | Solid | | Viscosity | | | | | |
|---|---|---|---|---|---|---|---|---|
| Sample | content (%) | Condition | 20° C. | | 30° C. | | 40° C. | |
| G8CN | 51.5 | After 1-day storage | 598 | cP | 450 | cP | 264 | cP |
| | | After 1-month storage | 2503 | cP | 1896 | cP | 1079 | cP |
| | | Increase | 319% | | 321% | | 309% | |
| G8CH1 | 51.6 | After 1-day storage | 767 | cP | 460 | cP | 268 | cP |
| | | After 1-month storage | 2877 | cP | 1949 | cP | 1104 | cP |
| | | Increase | 275% | | 324% | | 312% | |
| G8CH2 | 51.7 | After 1-day storage | 629 | cP | 458 | cP | 267 | cP |
| | | After 1-month storage | 2440 | cP | 1818 | cP | 1040 | cP |
| | | Increase | 288% | | 297% | | 290% | |
| G8CH4 | 52.8 | After 1-day storage | 700 | cP | 505 | cP | 289 | cP |
| | | After 1-month storage | 2536 | cP | 1865 | cP | 1065 | cP |
| | | Increase | 262% | | 269% | | 269% | |
| G8CH24 | 53.0 | After 1-day storage | 434 | cP | 322 | cP | 194 | cP |
| | | After 1-month storage | 795 | cP | 603 | cP | 336 | cP |

| | | | |
|---|---|---|---|
| Increase | 83% | 87% | 73% |

The increases in the viscosities between after 1-day and after 1-month cold storage became smaller with the enzymatic branching reaction time according to the present invention (Table 8). The smaller viscosity increase indicated that the enzymatic branching reaction stabilized the waxy maize maltodextrin solution against retrogradation during cold storage. The branched waxy maize maltodextrin solutions were also more transparent after cold storage than the unmodified waxy maize maltodextrin solution (data not shown).

In Vitro Starch Digestibility

The in vitro digestibilities of the unmodified and the branched waxy maize maltodextrins were analyzed using the method of Englyst et al. as mentioned in Example 1.

TABLE 9
In vitro digestibilities of unmodified and branched waxy maize
maltodextrins following the method of Englyst et al.

| | Free glucose and rapidly | Slowly digestible | Resistant |
|---|---|---|---|
| Samples | digestible starch (%) | starch (%) | starch (%) |
| G8CN | 90.4 | 4.7 | 4.9 |
| G8CH1 | 84.9 | 8.7 | 6.4 |
| G8CH2 | 80.1 | 5.9 | 14.0 |
| G8CH4 | 79.3 | 6.5 | 14.2 |
| G8CH24 | 70.3 | 4.6 | 25.1 |

In Table 9, the in vitro digestibility test of the unmodified and the branched waxy maize maltodextrins showed that the enzymatic branching reaction using transglucosidase and β-amylase according to the present invention resulted in an increasing proportion of the sum of SDS and RS with the increasing time of the enzymatic branching reaction, while reducing the sum of FG and RDS.

Conclusions

The DE values did not show obvious changes after the enzymatic branching reaction using transglucosidase and β-amylase according to the present invention although the amount of maltose in the waxy maize maltodextrin sample increased. The branched waxy maize maltodextrins were more stable against the retrogradation during cold storage as indicated by the smaller increase in the solution viscosity and opacity after prolonged cold storage. The in vitro digestibilities of the branched waxy maize maltodextrins showed that the enzymatic branching treatment with transglucosidase and β-amylase according to the present invention resulted in an increasing proportion of the sum of SDS and RS with the increasing duration of the enzymatic branching reaction time, while reducing the sum of FG and RDS.

### Example 3: Enzymatic Branching Modification of Maltodextrin Using Transglucosidase L “Amano” ® (from Amano Enzyme)

### Preparations

Branched Maltodextrins

Maltodextrin solutions (prepared using decarbonated water) with the concentrations of 57% to 67% solid contents were prepared using the tapioca maltodextrin TM42N mentioned in Example 1 and Glucidex 8C (Roquette) mentioned in Example 2. The maltodextrin was solubilized by constant stirring in a water bath at 55° C. for 30 min. Transglucosidase L “Amano” ® (from Amano Enzyme) and wheat β-amylase (Roquette) were added at the concentrations of 1 μL (or 1.1 U) and 0.1 μL (or 1.8 U) per gram dry maltodextrin, respectively. The mixture was incubated at 55° C. for 6 hours. The enzymes were deactivated by heating the samples in -boiling water for 20 min. The branched tapioca maltodextrins prepared at 57% and 67% solid contents were labelled as TM42S57 and TM42S67, respectively. Whereas, the branched waxy maize maltodextrins prepared at 57% and 62% solid contents were labelled as G8CS57 and G8CS62, respectively.

Analysis

Solid Content and Dextrose Equivalent

The solid contents of the maltodextrin solutions prepared for the enzymatic branching reaction following the present invention were analyzed based on the weight difference before and after overnight drying in an oven at 110° C.

The DE values of the resulting branched maltodextrins were analyzed and presented in Table 10.

TABLE 10
Solid contents of maltodextrin solutions for enzymatic
branching reaction and DE values and maltose contents
of the resulting branched maltodextrins.

| | Solid content during | | Maltose content |
|---|---|---|---|
| Sample | enzymatic branching | DE value | (% by weight) |
| TM42N | — | 17 | 3.51 |
| TM42S57 | 56.95% | 24 | 4.86 |
| TM42S67 | 67.42% | 21 | 5.18 |
| G8C | — | 8 | 1.38 |
| G8CS57 | 56.52% | 17 | 3.74 |
| G8CS62 | 61.89% | 16 | 1.99 |

The DE values of the branched maltodextrins increased after the enzymatic branching reaction using transglucosidase and β-amylase according to the present invention (Table 10). However, the DE values of the branched maltodextrins prepared at >60% solid content were lower than those of their counterparts prepared at <60% solid content.

Maltose Content

The maltose contents of the unmodified and the branched maltodextrins were analyzed using the same HPLC method as in Example 1. Each sample solution was diluted 50 times using 0.2% glycerol solution, and then filtered through a 0.45-μm membrane filter before being injected into the HPLC system.

Table 10 shows the amounts of maltose in the branched maltodextrin samples were higher than their unmodified maltodextrin counterparts. This was due to the hydrolysis reactions of β-amylase, increasing the DE values. For waxy maize maltodextrins, the amount of maltose was higher for G8CS57 than for G8CS62. It seems that β-amylase was less effective at higher solid content, probably due to the higher viscosity.

In Vitro Starch Digestibility

The in vitro digestibilities of the unmodified and the branched maltodextrins were analyzed using the method of Englyst et al. as mentioned in Example 1.

TABLE 11
In vitro digestibilities of unmodified and branched tapioca
maltodextrins following the method of Englyst et al.

| | | Rapidly | Slowly | |
|---|---|---|---|---|
| | Free glucose | digestible | digestible | Resistant starch |
| Samples | (%) | starch (%) | starch (%) | (%) |
| TM42N | 0.4 | 91.0 | 1.6 | 7.0 |
| TM42S57 | 4.0 | 74.2 | 7.8 | 14.0 |
| TM42S67 | 1.6 | 83.7 | 0.5 | 14.3 |
| G8C | 0.3 | 87.1 | 5.5 | 7.2 |
| G8CS57 | 3.9 | 81.3 | 5.8 | 9.1 |
| G8CS62 | 5.0 | 75.8 | 9.6 | 9.6 |

In Table 11, the in vitro digestibility test of the unmodified and the branched maltodextrins showed that the enzymatic branching reaction using transglucosidase and β-amylase according to the present invention resulted in an increased proportion of the sum of SDS and RS, while reducing the amount of RDS. TM42S57 had a larger proportion of the sum of SDS and RS than TM42S67. Furthermore, the change in the in vitro digestibilities of the waxy maize maltodextrin after the enzymatic branching reaction was smaller than those of the tapioca maltodextrin. This could be attributed to the higher viscosity of waxy maize maltodextrin, having lower DE value than the tapioca maltodextrin. At higher viscosity, such as at a lower DE or higher solid content, the enzymes have less mobility and therefore are less effective for the branching reaction.

Conclusions

The DE values of tapioca and waxy maize maltodextrins increased after the enzymatic branching reaction using transglucosidase and β-amylase according to the present invention, which could be attributed to the increase in the small sugars. The in vitro digestibility results showed that the enzymatic branching reaction using transglucosidase and β-amylase according to the present invention increased the sum of SDS and RS, while reducing the amount of RDS.

Regarding the effect of the concentration of maltodextrin on the enzymatic branching reaction, the higher concentration of solid should favor the branching (or condensation) reaction of transglucosidase over its hydrolytic reaction because hydrolysis requires water molecules. However, there was no clear trend observed with the solid content. This could be attributed to the viscosity of the maltodextrin solution. Higher viscosity will reduce the efficiency of the enzymes as the mobility is reduced. In addition, the changes were less obvious for waxy maize maltodextrin than for tapioca maltodextrin, which could be attributed to the higher viscosity of waxy maize maltodextrin, having lower DE value than the tapioca maltodextrin.

### Example 4: Enzymatic Branching Modification of Tapioca and Pea Maltodextrins Using Transglucosidase L “Amano” ® (from Amano Enzyme)

### Preparations

Tapioca and Pea Maltodextrins

Two types of starches were liquefied using Liquozyme Supra (a thermostable α-amylase from Novozymes) to produce maltodextrin. Tapioca and pea starches were used to prepare 35% and 30% starch slurry with demineralized water (pH 6.0 and 5.5), and the enzyme was added to the slurry at 3.0 and 3.5 μL/g dry starch, respectively. Each mixture was heated in a custom-built lab cooker at 110° C. and at an average flow rate of about 35 mL/min, which was about 17 min liquefaction time. The enzyme was then deactivated at 140° C. and the resulting maltodextrin solution was cooled to room temperature. The tapioca maltodextrin (TM18) had a DE of 18, and the pea maltodextrin (PM19) had a DE of 19. Both maltodextrin solutions had pH ˜5.5, and they were evaporated using a rotary evaporator (Rotavapor R-300, Buchi) to yield >50% solid content and used for further enzymatic branching reaction according to the present invention. The maltodextrins were stored in a refrigerator to prevent microbial growth.

Branched Maltodextrins

The solid content of each maltodextrin solution (pH ˜5.5) was adjusted and heated in a hot-water jacketed beaker at 90° C. for 30 min. The beaker was covered using aluminum foil to avoid the excessive water loss due to evaporation. After the maltodextrin solution had been cooled to 55° C., it was incubated with Transglucosidase L “Amano” ® (from Amano Enzyme) and wheat β-amylase (Roquette) at 55° C. The reaction conditions are summarized in Table 12. The enzymes were deactivated by heating the resulting branched maltodextrin back to 90° C. for 30 min.

TABLE 12
Conditions for the enzymatic branching reaction of tapioca and pea maltodextrins.

| | | Solid | β-amylase | | Transglucosidase | | Reaction |
|---|---|---|---|---|---|---|---|
| | Botanical | content | (U/g dry | | (U/g dry | Unit ratio | time |
| Sample | origin | (%) | maltodextrin) | | maltodextrin) | β-amylase:Transglucosidase | (h) |
| TM(22:1) | Tapioca | 50% | 7.2 | (0.4 μL) | 0.3 (0.3 μL) | 22:1 | 3 |
| TM(221:1) | Tapioca | 50% | 72.0 | (4 μL) | 0.3 (0.3 μL) | 221:1 | 3 |
| TM(1:10) | Tapioca | 45% | 0.2 | (0.01 μL) | 1.6 (1.5 μL) | 1:10 | 24 |
| TM(1:1) | Tapioca | 50% | 1.8 | (0.1 μL) | 1.6 (1.5 μL) | 1:1 | 6 |
| PM(1:1) | Pea | 50% | 1.8 | (0.1 μL) | 1.6 (1.5 μL) | 1:1 | 6 |

Analysis

Dextrose Equivalent and Degree of Branching

The DE values of the unmodified and the branched maltodextrins were analyzed following the method of Bertrand (Bulletin de la Société Chimique de France, 1906, 35 pp. 1285-1299).

The amounts of α-1,4 and α-1,6 glycosidic linkages in the unmodified and the branched maltodexrins were analyzed by ¹H NMR technique using 5-mm NMR tubes with an Avance III Fourier transform spectrometer (Bruker Spectrospin) operating at 400 MHz and 60° C. The procedure is as follows. Each sample (10 mg) was dissolved in 0.75 mL D₂O (min. 99%, Eurisotop) in a water bath. After being cooled to room temperature, 50 μL solution (10 mg/g) of sodium salt of 3-trimethylsilyl-1-propanesulfonic acid (Aldrich) was added to each sample. Without solvent suppression, the acquisition was performed at a relaxation time of at least 10 s and without rotation. The spectrum was collected after Fourier transformation, phase correction and subtraction of the base line in manual mode (without exponential multiplication; LB=GB=0).

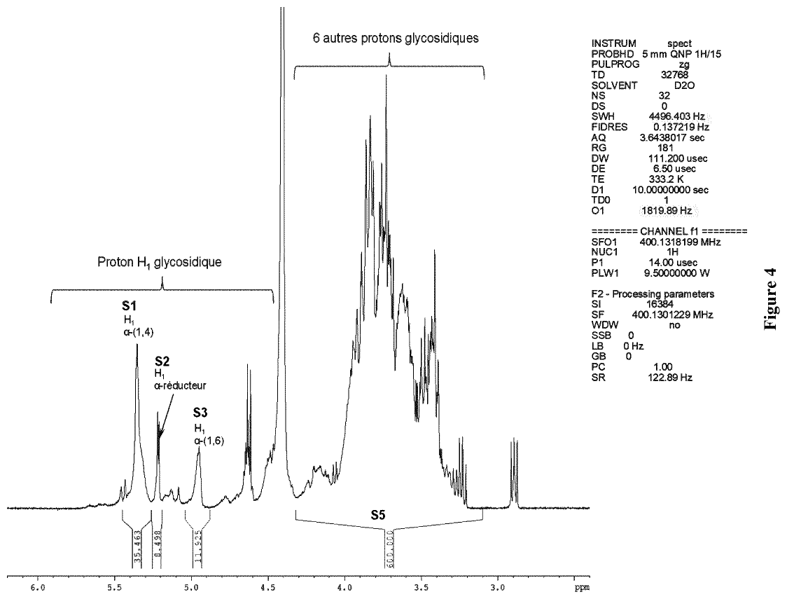

The integration of the signals (see FIG. 4 and Table 13) was performed as follows. The S5 signal was normalized to 600. This signal corresponded to the non-exchangeable protons of an anhydroglucose unit (H₂, H₃, H₄, H₅ and 2H₆). The signals S1, S2, and S3 corresponded to H₁ of α-1,4 linkage, H₁ of reducing end with a conformation, and H₁ of α-1,6 linkage, respectively. The S4 signal, which corresponded to reducing end with β conformation, was estimated by multiplying S2 with 1.5. The S6 signal was estimated by subtraction method S6=100−(S1+S2+S3+S4).

TABLE 13
Signal determination of ¹H NMR spectrum.

| Integrated | Limits of integration | | |
|---|---|---|---|
| surface area | (in ppm) | | Types of bonds |
| S1 | 5.45 | 5.26 | H₁ α-(1,4) |
| S2 | 5.26 | 5.19 | H₁ α-reducers |
| S3 | 5.04 | 4.88 | H₁ α-(1,6) |
| S5 | 4.32 | 3.10 | Other protons (H₂, H₃, H₄, |
| | | | etc.) i.e. 6 protons |

The proportions of α-1,4 and α-1,6 linkages were determined from the surface areas of S1 and S3 signals, and the summation of the two linkages was normalized to 100 in order to express them in percentage. Thus, the degree of branching was calculated as the amount of α-1,6 glycosidic linkages divided by the sum of α-1,4 and α-1,6 glycosidic linkages.

TABLE 14
DE values and degree of branching of unmodified
and branched maltodextrins.

| Samples | DE value | Degree of branching (%) |
|---|---|---|
| TM18 | 18 | 5.0 |
| PM19 | 19 | 4.8 |
| TM(22:1) | 30 | 7.3 |
| TM(221:1) | 38 | 8.2 |
| TM(1:10) | 35 | 38.8 |
| TM(1:1) | 25 | 14.0 |
| PM(1:1) | 22 | 16.7 |

In Table 14, the DE values of TM(22:1), TM(221:1), and TM(1:10) had high DE values above 30 although the first two samples were only incubated for 3 h, whereas TM(1:10) was incubated for 24 h. This could be explained by the high ratio of β-amylase to substrate used to produce TM(22:1) and TM(221:1), releasing high amount of maltose and increasing the number of reducing sugars in the solution. The DE values of TM(1:1) and PM(1:1) were lower than TM(1:10), probably due to the reaction times of TM(1:1) and PM(1:1) were shorter, resulting in lower degree of hydrolysis.

Table 14 also summarizes the degree of branching of the unmodified and the branched maltodextrins. The high concentration of β-amylase only increased the degree of branching of TM(22:1) and TM(221:1) to 8% from 5% in the unmodified tapioca maltodextrin. Using a lower concentration of β-amylase, the degree of branching of tapioca maltodextrins increased up to 39% although the DE value were lower than that of TM(221:1). The results indicate that a smaller amount of β-amylase with a higher amount of transglucosidase favors the branching reaction, whereas the opposite favors the hydrolysis reaction.

In Vitro Starch Digestibility

The in vitro digestibilities of the unmodified and the branched maltodextrins were analyzed using the method of Englyst et al. as mentioned in Example 1.

TABLE 15
In vitro digestibilities of unmodified and branched
maltodextrins following the method of Englyst et al.

| | | Rapidly | Slowly | |
|---|---|---|---|---|
| | Free glucose | digestible | digestible | Resistant starch |
| Samples | (%) | starch (%) | starch (%) | (%) |
| TM18 | 2 | 85 | 6 | 7 |
| PM19 | 2 | 91 | 0 | 7 |
| TM(22:1) | 3 | 96 | 7 | 0 |
| TM(221:1) | 3 | 87 | 4 | 6 |
| TM(1:10) | 15 | 46 | 9 | 30 |
| TM(1:1) | 6 | 83 | 10 | 1 |
| PM(1:1) | 4 | 74 | 5 | 17 |

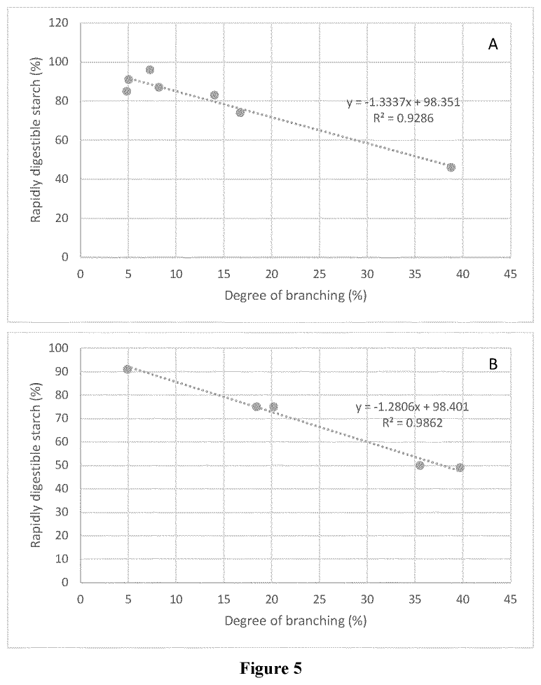

Table 15 summarizes the in vitro digestibility results of the unmodified and the branched maltodextrins. TM(22:1) and TM(221:1) had RDS content higher than the unmodified tapioca starch (TM18), suggesting that the high ratio of β-amylase to substrate resulted in small molecules that were more rapidly digestible than the molecules in the unmodified tapioca maltodextrin (TM18). On the other hand, TM(1:10) contained the lowest RDS among all treatments. Whereas TM(1:1) and PM(1:1) showed slight decreases in the RDS content compared with their unmodified maltodextrin counterparts. Similar to the other examples, the RDS content was negatively correlated with the sum of SDS and RS. The RDS content was also negatively correlated with the degree of branching (FIG. 5A), suggesting that the slow digestion properties of branched maltodextrins is due to the higher amount of α-1,6 glycosidic linkages that cannot be hydrolyzed by pancreatic α-amylase and may pose as a steric hindrance for the branched maltodextrin substrate to bind with pancreatic α-amylase.

Free Sugar Profile

The free sugar profile were analyzed using HPAEC system (Dionex™ ICS-5000, Thermo Scientific™) with pulsed amperometric detection (PAD). The column used for the analysis was CarboPac™ PA1 (4*250 mm), preceded by a guard column CarboPac™ PA1 (4*50 mm). Each samples was accurately weighed, mixed with 1.0 mL internal standard (melibiose), and diluted with 20 mL distilled water. The mixture was stirred for 10 min and filtered through a 0.45-μm membrane. The injection volume was 5 μL. The sample was eluted at 30° C. in gradient mode of NaOH 100 mM (A) and NaOH 100 mM+CH₃COONa 500 mM (B), which was programmed as follows: 2% B at 0.0 min, 5% B at 60.0 min, 30% B at 65.0 min, 100% B at 65.05 min, 100% B at 75.0 min, 2% B at 75.05 min, and 2% B at 90.0 min.

TABLE 16
Free sugar profile of unmodified and branched maltodextrins.

| | Glucose | Maltose | Isomaltose | Maltotriose |
|---|---|---|---|---|
| Samples | (%) | (%) | (%) | (%) |
| TM18 | 0.8 | 4.1 | <0.1 | 6.5 |
| PM19 | 1.1 | 4.2 | <0.1 | 4.4 |
| TM(22:1) | 1.4 | 19.2 | <0.1 | 10.6 |
| TM(221:1) | 1.6 | 26.5 | <0.1 | 10.6 |
| TM(1:10) | 7.2 | 1.1 | 5.5 | 0.4 |
| TM(1:1) | 3.3 | 4.9 | 1.3 | 3.6 |
| PM(1:1) | 3.9 | 3.0 | 1.9 | 2.5 |

The free sugar profiles of the unmodified and the branched maltodextrins are summarized in Table 16. Maltodextrin normally has higher amounts of maltose and maltotriose than that of glucose because α-amylase mainly hydrolyzes starch into maltose and maltotriose, instead of glucose. TM(22:1) and TM(221:1) had the highest amount of maltose due to the reaction of β-amylase, which hydrolyzes starch from the non-reducing ends0 and each successful hydrolysis releases a maltose. This agrees with their high DE values (Table 14). On the other hand, the maltose contents of TM(1:1) and PM(1:1) were similar or slightly lower than those of their unmodified maltodextrin counterparts. In addition, TM(1:10) had the lowest amount of maltose. These results confirm that the maltose produced by β-amylase was mostly used by the transglucosidase for the branching reaction. The branching reaction of transglucosidase also released glucose, increasing the glucose contents in these samples along with isomaltose, which is another product of transglucosidase.

Conclusions

The DE values of tapioca and pea maltodextrins increased after the enzymatic branching reaction using transglucosidase and β-amylase, which could be attributed to the increase in the small sugars. A high ratio of β-amylase to maltodextrin substrate and a high enzyme activity unit ratio of β-amylase to transglucosidase favored the hydrolysis of maltodextrin from the non-reducing ends, releasing a high amount of maltose. As the results, the DE value was increased rapidly in a short time, while the degree of branching was only slightly increased. The products of β-amylase hydrolysis seemed to be more rapidly digestible than the molecules in the unmodified maltodextrin.

On the other hand, a low ratio of β-amylase to maltodextrin substrate combined with a low enzyme activity unit ratio of β-amylase to transglucosidase, following the present invention, favored the branching reaction of maltodextrin indicated by the increased amount of α-1,6 glycosidic linkages. The α-1,6 glycosidic linkages cannot be hydrolyzed by pancreatic α-amylase and may pose as a steric hindrance for the branched maltodextrin substrate to bind with pancreatic α-amylase, resulting in lower digestion rate or a lower amount of RDS as shown by the test results obtained using the method of Englyst et al.

### Example 5: Comparison of Two Transglucosidases for Enzymatic Branching Modification of Tapioca Maltodextrin

### Preparations

Tapioca Maltodextrin

Tapioca maltodextrin (TM17) was prepared similar to Example 4. Tapioca starch slurry (35% solid content in demineralized water, pH 6.0) was liquefied using Liquozyme Supra (Novozymes, 2.9 μL/g dry starch) in a custom-built lab cooker at 110° C. and at an average flow rate of about 35 mL/min, which was about 17 min liquefaction time. The enzyme was then deactivated at 140° C., and the resulting maltodextrin solution was cooled to room temperature. The tapioca maltodextrin had a DE of 17 and pH 5.6, and it was then evaporated using a rotary evaporator (Rotavapor R-300, Buchi) to yield >50% solid content and used for further enzymatic branching reaction according to the present invention. The maltodextrin was stored in a refrigerator to prevent microbial growth.

Branched Maltodextrins

The solid content of maltodextrin solution (pH ˜5.5) was adjusted to around 47% and heated in a hot-water jacketed beaker at 90° C. for 30 min. The beaker was covered using aluminum foil to avoid the excessive water loss due to evaporation. After the maltodextrin solution had been cooled to 55° C., it was incubated with two enzyme systems. The first system employed Tranglucosidase L-2000® from DuPont, and the second system employed Transglucosidase L “Amano” ® from Amano Enzyme. The reaction was carried at 55° C. for 6 or 24 hours, which is summarized in Table 17. The enzymes were deactivated by heating the resulting branched maltodextrin back to 90° C. for 30 min.

TABLE 17
Conditions for the enzymatic branching reaction of tapioca maltodextrins.

| | β-amylase | | Transglucosidase | | | Reaction |
|---|---|---|---|---|---|---|
| | (U/g dry | | (U/g dry maltodextrin) | | Unit ratio | time |
| Sample | maltodextrin) | | Amano | DuPont | β-amylase:Transglucosidase | (h) |
| TM-A6 | 0.2 | (0.01 μL) | 1.6 (1.5 μL) | 0.0 | 1:10 | 6 |
| TM-A24 | 0.2 | (0.01 μL) | 1.6 (1.5 μL) | 0.0 | 1:10 | 24 |
| TM-D6 | 1.8 | (0.1 μL) | 0.0 | 1.6 (1.0 μL) | 1:1 | 6 |
| TM-D24 | 1.8 | (0.1 μL) | 0.0 | 1.6 (1.0 μL) | 1:1 | 25 |

Analysis

Dextrose Equivalent and Degree of Branching

The DE values of the unmodified and the branched tapioca maltodextrins were analyzed following the method of Bertrand as mentioned in Example 4. The amounts of α-1,4 and α-1,6 glycosidic linkages in the unmodified and the branched tapioca maltodextrins were analyzed by ¹H NMR technique as mentioned in Example 4.

TABLE 18
DE values and degree of branching of unmodified
and branched tapioca maltodextrins.

| Samples | DE value | Degree of branching (%) |
|---|---|---|
| TM17 | 17 | 4.9 |
| TM-A6 | 24 | 18.4 |
| TM-A24 | 37 | 35.5 |
| TM-D6 | 29 | 20.2 |
| TM-D24 | 39 | 39.7 |

Table 18 shows that both DE value and degree of branching of the tapioca maltodextrin increased with the reaction time, indicating that the enzymatic branching reaction occurred longer than 6 hours. The increase in the DE value was due to the production of maltose by β-amylase, which was then used by the transglucosidase for the branching reaction, releasing glucose as the by-product. At the same reaction time, both enzyme systems produced comparable DE values and degrees of branching albeit Transglucosidase L-2000® (from DuPont) showing slightly higher DE value and degree of branching. These results suggest that both enzyme systems were effective for branching tapioca maltodextrin.

In Vitro Starch Digestibility

The in vitro digestibilities of the unmodified and the branched tapioca maltodextrins were analyzed using the method of Englyst et al. as mentioned in Example 1.

TABLE 19
In vitro digestibilities of unmodified and branched tapioca
maltodextrins following the method of Englyst et al.

| | | Rapidly | Slowly | |
|---|---|---|---|---|
| | Free glucose | digestible | digestible | Resistant starch |
| Samples | (%) | starch (%) | starch (%) | (%) |
| TM17 | 2 | 91 | 4 | 3 |
| TM-A6 | 7 | 75 | 8 | 10 |
| TM-A24 | 14 | 50 | 7 | 29 |
| TM-D6 | 7 | 75 | 10 | 8 |
| TM-D24 | 16 | 49 | 13 | 22 |

Table 19 summarizes the in vitro digestibility results of the unmodified and the branched tapioca maltodextrins. The amount of RDS decreased with the reaction time, whereas the amount of FG and the sum of SDS and RS increased, showing that the branching reaction decreased the digestion rate of the maltodextrin. Indeed, there is a strong negative correlation between the degree of branching and the RDS content (FIG. 5B).

Similar to the DE and the degree of branching (Table 18), at the same reaction time, the two enzyme systems produced branched maltodextrins with comparable in vitro digestion profile, indicating that both transglucosidases were effective to branch tapioca maltodextrin and reduce its digestion rate.

### Free Sugar Profile

The free sugar profiles of the unmodified and the branched tapioca maltodextrins were analyzed as mentioned in Example 4.

TABLE 20
Free sugar profile of unmodified and branched maltodextrins.

| | Glucose | Maltose | Isomaltose | Maltotriose | Panose |
|---|---|---|---|---|---|
| Samples | (%) | (%) | (%) | (%) | (%) |
| TM17 | 0.5 | 3.9 | <0.1 | 5.7 | <0.1 |
| TM-A6 | 3.2 | 2.6 | 1.7 | 1.5 | 3.3 |
| TM-A24 | 7.6 | 1.3 | 5.7 | 0.3 | 2.2 |
| TM-D6 | 3.5 | 6.8 | 1.6 | 3.4 | 6.0 |
| TM-D24 | 8.3 | 2.3 | 6.3 | 0.3 | 3.9 |

The free sugar profiles of the unmodified and the branched tapioca maltodextrins are summarized in Table 20. The glucose and isomaltose contents increased with the incubation time as these are the products of transglucosidase. The contents of maltose, maltotriose, and panose decreased between 6-hour and 24-hour incubation, indicating that transglucosidase can use these molecules as its substrates. In addition, the contents of isomaltose were still less than 7% in the branched tapioca maltodextrin samples after 24-hour incubation, and therefore isomaltose was not the main molecule responsible for the slow digestion properties. Overall, the amounts of these small sugars remained low after 24-hour incubation, which differentiates the present invention from IMOS.

Conclusions

Tapioca maltodextrin was branched using either Tranglucosidase L-2000® from DuPont or Transglucosidase L “Amano” ® from Amano Enzyme in combination of wheat β-amylase. The reaction was performed for 6 or 24 hours. The DE value, the degree of branching, the amount of glucose, and the sum of SDS and RS of the tapioca maltodextrin increased with the increasing of reaction time, whereas the amount of RDS decreased. The amounts of glucose, maltose, isomaltose, maltotriose, and panose remained low after 24-hour incubation. The results indicated that the increase in the degree of branching reduced the digestion rate of the maltodextrin. The increase in the DE value was due to release of maltose by β-amylase, which was then used by the transglucosidase for the branching reaction, releasing glucose as the by-product. It also seems that transglucosidase could use maltotriose and panose as its substrates. At the same reaction time, both enzyme systems produced branched tapioca maltodextrins with comparable DE values, degrees of branching, and digestion profile, indicating that both enzyme systems were effective to branch tapioca maltodextrin and reduce its digestion rate.

1. A method for preparing a composition comprising branched starch hydrolysate, comprising the step of incubating a composition comprising starch hydrolysate with a mixture of maltose-generating amylase and transglucosidase enzymes in enzyme activity unit ratio of 9:5 to 1:20, advantageously of 9:5 to 1:10, preferably 10:6 to 1:2, even more preferably around 3:2 to 1:1.

2. The method according to claim 1, wherein the composition compnsmg starch hydrolysate is obtained from a starch selected from tapioca, waxy tapioca, maize, waxy maize, rice, waxy rice, wheat, waxy wheat, barley, waxy barley, sorghum, waxy sorghum, potato, waxy potato, sweet potato, waxy sweet potato, sago, millet, mung bean, pea, faba bean, chickpea, arrowroot, buckwheat, quinoa, lotus root, preferably tapioca, waxy maize, or pea.

3. The method according to claim 1, wherein the composition comprising starch hydrolysate is obtained by incubating said starch source with one or multiple amylases, such as thermostable α-amylase.

4. The method according to claim 1, wherein the DE of the starch hydrolysate is between 1 and 30, preferably between 5 and 20, even more preferably between 8 and 15.

5. The method according to claim 1, wherein the free maltose content of the starch hydrolysate is less than 15%, preferably less than 10%, even more preferably less than 5% by weight.

6. The method according to claim 1, wherein the viscosity of the composition comprising starch hydrolysate prior to incubation is below 5000 cP, more preferably below 2000 cP at 50% solid content at 25° C.

7. The method according to claim 1, wherein the maltose-generating amylase is selected from the group consisting of maltogenic α-amylases and maltogenic β-amylases, preferably β-amylase, even more preferably wheat β-amylase.

8. The method according to claim 1 wherein the step of incubating the composition comprising starch hydrolysate with a mixture of maltose-generating amylase and transglucosidase enzymes is carried out at a temperature comprised between 40° C. and 70° C., preferably between 50° C. and 60° C., even more preferably around 55° C.

9. The method according to claim 1, wherein the step of incubating the composition comprising starch hydrolysate with a mixture of maltose-generating amylase and transglucosidase enzymes is carried out for a duration comprised between 1 and 72 hours, preferably between 2 and 24 hours, even more preferably 4 to 8 hours.

10. The method according to claim 1, wherein the composition comprising starch hydrolysate has a solid content comprised between 30% and 70% by weight of the total composition, preferably between 40% and 60% by weight of the total composition, even more preferably between 45% and 55% by weight of the total composition.

11. The method according to claim 1, wherein the step of incubating the composition comprising starch hydrolysate with mixture of maltose-generating amylase and transglucosidase enzymes is carried out at pH value comprised between 4 and 8, preferably between 4.5 and 7, even more preferably between 5 and 6.5

12. The method according to claim 1, wherein the transglucosidase is a D-glucosyltransferase.

13. The method according to claim 1, wherein the concentration of free maltose is monitored during incubation and is preferably less than 12%, preferably less than 8%, even more preferably less than 5% by weight of the composition.

14. A composition comprising branched starch hydrolysate obtainable by the method according to claim 1.

15. A composition comprising branched starch hydrolysate wherein said composition:
a) has a DE value between 10 and 50, preferably between 12 and 30, even preferably between 15 and 25,
b) has a percentage of α-1,6 linkages of at least 7, preferably of at least 8%, at least 9%, at least 10%, at least 11%, at least 12%, at least 13%, at least 14%, at least 15%, at least 16%, at least 17%, at least 18%, at least 19%, preferably of at least 20%, at least 21%, at least 22%, at least 23%, at least 24%, at least 25%, and even more preferably of at least 30%,
c) comprises at least 30% by weight, preferably at least 35%, preferably at least 40% by weight, even more preferably at least 43% by weight of branches of the branched starch hydrolysate with a DP equal to or greater than 10.

16. Food product comprising a composition according to claim 14, such as for diabetic meals and meal replacement, energy gel, solid beverages, and sports beverages.
